(19) United States
(12) Patent Application Publication
(10) Pub. No.: US 2020/0273541 A1
(43) Pub. Date: Aug. 27, 2020

(54) UNSUPERVISED PROTEIN SEQUENCE GENERATION

(71) Applicant: THE REGENTS OF THE UNIVERSITY OF CALIFORNIA, Oakland, CA (US)

(72) Inventors: Zachary Costello, Berkeley, CA (US); Hector Garcia Martin, Oakland, CA (US)

(21) Appl. No.: 16/803,768

(22) Filed: Feb. 27, 2020

Related U.S. Application Data
(60) Provisional application No. 62/811,443, filed on Feb. 27, 2019.

(51) Int. Cl. G16B 40/30; G16B 40/20; G16B 20/00; G16B 30/00; G16B 50/00; G06N 20/00

(52) U.S. Cl. CPC G16B 40/30; G16B 40/20; G06N 20/00; G16B 30/00; G16B 50/00; G16B 20/00

(57) ABSTRACT

A method of unsupervised protein sequence generation includes determining a dataset of known protein sequences, wherein the dataset comprises unlabeled or sparsely labeled data. The method further includes training, by a processing device, a generative model on the dataset. The method further includes generating, using the generative model, a semantically-valid protein sequence example based on the dataset.

### CROSS-REFERENCE TO RELATED APPLICATIONS

This application claims the benefit of U.S. Provisional Patent Application No. 62/811,443, filed on Feb. 27, 2019, the entire contents of which are incorporated by reference herein.

### STATEMENT OF GOVERNMENT SUPPORT

This invention was made with government support under Contract No. DE-AC02-05CH11231 awarded by the U.S. Department of Energy. The government has certain rights in this invention.

### SEQUENCE LISTING

The instant application contains a sequence listing which has been submitted in ASCII Format via EFS-Web and is hereby incorporated by reference in its entirety. Said ASCII copy, created on Apr. 29, 2020, is named L102142_1350US_1_SEQ_LISTING_ST25.txt and is 38,182 bytes in size.

### FIELD

The present invention relates to the protein sequence generation, and more particularly, relates to unsupervised protein sequence generation.

### BACKGROUND

Proteins are the main functional unit of life, performing a majority of tasks within the cell. Each one is uniquely defined by a sequence of amino acids. These macromolecules perform a diverse set of functions including catalysis, structural support, mechanical transduction, molecular transport, and sensing. The ability to reliably engineer proteins with a specified function in a systematic way would be transformative for synthetic biology, allowing for the explicit design of molecular machines with a targeted function for a diverse array of applications.

### BRIEF DESCRIPTION OF THE DRAWINGS

FIG. 1 illustrates architecture of a variational autoencoding model, according to one embodiment.

FIG. 2 is a block diagram that illustrates reconstruction accuracy of a variational autoencoding model, according to one embodiment.

FIG. 3 is a block diagram that illustrates mean and variance of latent space components, and a heat map for a block of the covariance matrix, according to one embodiment.

FIG. 4 is a block diagram that illustrates accuracy of a variational autoencoding model, according to one embodiment.

FIG. 5 is a block diagram that illustrates accuracy of a variational autoencoding model, according to one embodiment.

FIG. 6 is a block diagram that illustrates cross-validated performance of a variational autoencoding model, according to one embodiment.

FIG. 7 is a flow diagram illustrating unsupervised protein sequence generation, according to one embodiment.

FIG. 8 is a block diagram of an example apparatus that may perform one or more of the operations described herein, in accordance with one embodiment.

### DETAILED DESCRIPTION

Unsupervised protein sequence generation is described herein. In particular, in one embodiment, an approach to protein design and phenotypic inference using a generative model for protein sequences is described.

Proteins are the main functional unit of life, performing a majority of tasks within the cell. Each protein is uniquely defined by a sequence of amino acids. These macromolecules perform a diverse set of functions including catalysis, structural support, mechanical transduction, molecular transport, and sensing. The ability to reliably engineer proteins with a specified function in a systematic way would be transformative for synthetic biology, allowing for the explicit design of molecular machines with a targeted function for a diverse array of applications.

In some embodiments, not every protein sequence encodes a functional protein. It has been estimated that randomly selecting a protein sequence would produce a functional protein about one time in a million. In general, folding (e.g., functioning) protein sequences appear to be rare in the space of all possible sequences. As such, there is an underlying syntax to these sequences that is necessary for function to be present. Syntactic correctness gives rise to recognized secondary (e.g., alpha helices) and tertiary structures (e.g., alpha/beta-barrel domains), which in aggregate may lead to function. Though large quantities of sequence data exist, this syntax may not be currently understood well enough to explicitly perform design without structural knowledge or an existing protein as a starting point.

Described herein is a novel technique, which can generate syntactically correct proteins that are likely (e.g., have a high likeliness of success above a defined threshold) to fold and function using only sequence data. Further, the embodiments described herein can be used as a design tool to generate novel proteins which are likely to have a specified or defined set of properties or functions.

Protein engineering has enabled the creation of an array of novel and useful proteins. Metabolic enzymes and pathways were developed for metabolic engineering. Promising cancer therapeutics have been developed. Biosensors have been designed for rapid detection of various biomolecules. Further, catalysts were designed to accelerate organic chemistry syntheses. While there have been successes, engineering proteins with a desired phenotype has remained a difficult task that requires expert level skill to perform successfully.

Even under the best conditions, protein engineering is costly and time consuming. Design tasks in protein engineering may require solving the inverse problem of finding a sequence that will impart a specific function to a protein. In the field of protein engineering, two broad categories of methods may be used: directed evolution and de novo design. These approaches may be used separately or in a complementary fashion. In one embodiment, directed evolution approaches aim to iteratively enrich for a desired function through stages of mutation and selection of an initial protein sequence. Such approaches utilize one or more starting proteins that can reasonably be evolved to have the desired function. These approaches are advantageous in some aspects, because they do not require understanding of the relationship between sequence and function, and they can still reach desired performance characteristics in a systematic way. An important limitation of these methods is that they require a protein starting point that is able to be evolved to a desired function.

De novo methods use the principals of protein folding to design sequences with structure that results in a chosen function. Determining the structure of a protein with the function of interest may be a reasonable task for a human designer. De novo methods may then find sequences that are likely to have the structure of interest. This approach is distinguished from directed evolution by attempting to understand the relationship between sequence and function, mediated through protein structure. Because of this, de novo techniques may not be restricted to portions of the protein sequence space that have already been explored by nature.

Described herein is a novel, structure-free (e.g., does not use protein structure), approach to protein design and property inference using a deep generative model. This model may be augmented by a semi-supervised approach for downstream design, classification, or regression tasks. The embodiments described herein allow for the building and execution of a model that intuits the underlying rules implicit in the structure of natural proteins. Advantageously, this allows for the use of the model, which understands the syntax of protein construction, as a tool to understand protein properties and to design function.

This approach has substantial benefits over both directed evolution and de novo methods. Because structure is not used to train the underlying model, much larger data sets are available for training, with over 140 million protein sequences publicly accessible on the UniProt database, for example. This allows for the training of more accurate models than would be possible with the approximately 150 thousand structures publicly available on the protein database. Furthermore, this model encodes proteins into a feature space which is useful for downstream tasks.

In various embodiments, generative models may be successfully applied to many other domains where unlabeled or sparsely labeled data is abundant. Generative models are able to take a collection of unlabeled examples of a particular type of data and use it to create novel, semantically-valid examples from that data set. Such models may also be used to perform unsupervised language translation, and design dental implants. Currently, generative models are classified as variational autoencoders, generative adversarial networks, or normalizing flows. The advantages and disadvantages must be weighed when choosing one for a particular application. Although a variational autoencoder that can both encode protein sequences into a useful feature space and generate valid protein examples from that space is primarily described herein for convenience, any other type of generative model may be used.

Variational autoencoders have several properties that make them well suited for protein engineering applications. Variational autoencoders learn a useful latent feature space where any protein sequence can be mapped. In one embodiment, the feature vectors are organized into regions of similar homology due to the optimization constraints so that similar sequences are encoded close in feature space. The set of all vectors in this data set may be constrained to be distributed in a multivariate standard normal distribution. Advantageously, this constraint makes sampling efficient. Additionally, these models have the ability to generate examples of novel proteins by reconstructing points in the feature space that are not explicitly occupied by samples from the data set. These models estimate the underlying joint distribution between amino acid residues in a protein sequence, allowing for modeling of all possible interactions that occur between amino acid residues.

While supervised learning, or generative models used to encode RNA expression profiles, may generate desired results, the unsupervised embodiments described herein advantageously use the entire known proteome to train the model. Training on a data set that is substantially complex introduces substantially more considerations into model architecture. Additionally, unsupervised models have not been used as a design tool to generate new sequences. The embodiments described herein provide for methods and systems for encoding all of the known protein space, instead of specific families of proteins, so that one can intuit the general structure of the entirety of protein sequence space.

Technical implementation details of BioSeqVAE, an unsupervised protein sequence generation model, are described herein. The trained model, resulting from BioSeqSAE, may be applied to a set of downstream tasks, demonstrating its usefulness to various design, classification, and regression problems important to protein engineering. Advantageously, BioSeqVAE is able to, among other tasks: (i) handle sequences with variable lengths; (ii) model interactions between distant amino acid residues; (iii) utilize a useful latent feature space; and (iv) generate realistic protein sequences.

### Data Sources and Processing

In one embodiment, the data to train BioSeqVAE may be acquired from the UniProt database. In other embodiments, any other suitable database may be used. The UniProt sequence database may be separated into two separate parts: SwissProt and TREMBL. The SwissProt Database is hand curated and contains about 550 thousand proteins. The TREMBL part of the Database is computationally predicted and contains approximately 140 million sequences. Since a goal of this model is to learn the general structures of protein sequences, representative sequences from clusters of proteins with similar homology may be included. In one embodiment, sequences in the database may be clustered into groups that share over a defined threshold (e.g., 80%) homology. Then one sequence may be chosen per cluster. This operation may be performed using the Linclust command line tool, or any other suitable tool. Sequences may be further pruned by selecting sequences between 100 and 1000 amino acid residues in length. In other embodiments, other ranges may be used. The data cleaning operation may reduce the SwissProt and TREMBL datasets to 200 thousand and 45 million sequences respectively. In one embodiment, models may be trained only on the SwissProt dataset. In other embodiments, any other dataset, or combination of datasets, may be used. The sequences may be represented with one hot encoding with 21 categories, where 20 categories were amino acids and one category represented sequence end, for example. In other embodiments, any other number and classification of categories may be used.

### Model Architecture

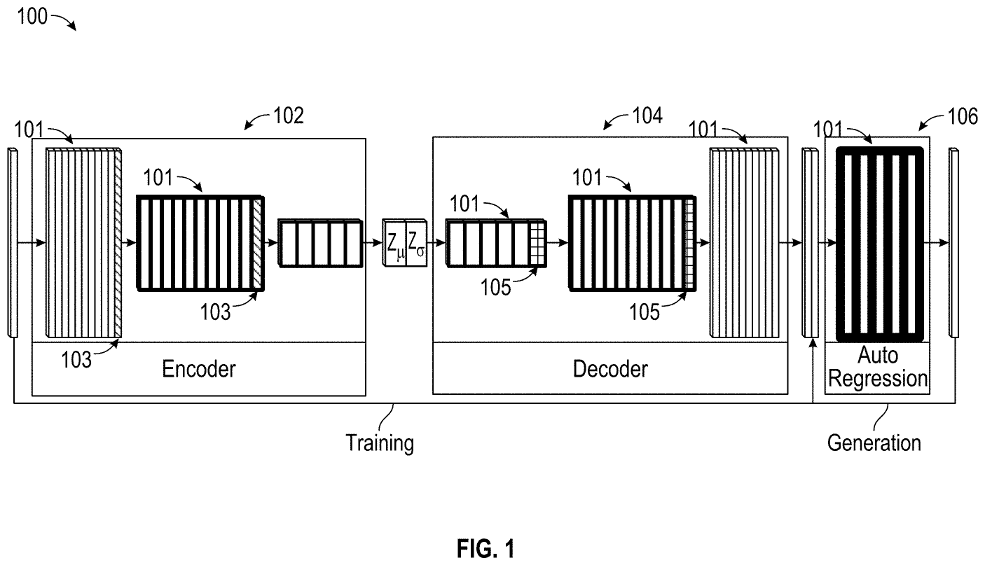

In one embodiment, a modified variational autoencoding model 100 may be used to perform unsupervised learning on protein sequences, as illustrated in FIG. 1. In one embodiment, model 100 may construct some dataset by taking N samples x˜X. In one embodiment, this is the set of all known protein sequences after the data cleaning protocol from above is performed. One objective may be to maximize the likelihood of the model pθ(x) given the data. In general, this objective function is intractable to evaluate:

$${max}_{θ} log {p}_{θ} ( x ) . \qquad ( 1 )$$

One advantage of the variational autoencoder described herein, is that a set of latent variables z ∈ R^{m} may be introduced, and the model may be separated into two components. In one embodiment, there may be an encoder q_{φ}(z|x) 102, which estimates the latent variable z given a particular data point x and a decoder pθ(x|z) 104, which produces an output in data space x given a particular point in the latent space z. Both the encoder 102 and decoder 104 may be deep learning models parameterized by their respective weights θ and φ. Starting from the objective function of the optimization problem in (1), a computationally tractable lower bound may be derived on the objective using Jensen's Inequality, as shown below:

$$\begin{matrix}log {p}_{0} ( x ) = log {\int }_{- ∞}^{∞} {p}_{θ} ( x , z ) dz \\ = log \int {p}_{θ} ( x , z ) \frac{{q}_{φ} ( z \vert x )}{{q}_{φ} ( z \vert x )} dz \\ = log {𝔼}_{{q}_{φ} ( z \vert x )} [ \frac{{p}_{θ} ( x \vert z )}{{q}_{φ} ( z \vert x )} ] \\ \geq {𝔼}_{{q}_{φ} ( z \vert x )} [ log {p}_{θ} ( x , z ) ] - {𝔼}_{{q}_{φ} ( z \vert x )} [ log {q}_{φ} ( z \vert x ) ]\end{matrix}$$

Here, instead of explicitly maximizing the likelihood of the model, a lower bound on that objective may be optimized. This lower bound may be described as the evidence lower bound [ELBO]. Using the definition of the KL divergence, the objective can be rewritten in an easier to interpret form:

$${ℒ}_{ELBO} ( θ , φ , x ) = {𝔼}_{{q}_{x} ( z \vert x )} [ log {p}_{θ } ( x \vert z ) ] - {D}_{KL} ( {q}_{φ} ( z \vert x ) \vert \vert p ( z ) ) .$$

The ELBO loss as expressed above has two terms with straightforward interpretations. The first term is the reconstruction loss, which measures how well a particular data point is reconstructed when run through both the encoder 102 and decoder 104. The second term is the closeness of the latent space to a chosen prior distribution. In one embodiment, the prior distribution may be selected to be a standard multivariate normal distribution. This makes sampling points from the distribution of protein sequences efficient, because points in the latent feature space may be sampled from the standard normal distribution and used to generate corresponding protein sequences in the data distribution. For protein sequence design and phenotypic inference, both an accurate reconstruction and an informative latent space may be desired. To this end, a high capacity decoder may be chosen to encourage high reconstruction accuracy. Several enhancements may be performed to help make the latent space encode informative features by constraining the amount of mutual information between x and z in the encoding model. The result may be used to augment the ELBO objective and force the model to encode information in the latent space. In one embodiment, the resulting objective may have the form:

$${ℒ}_{INFOVAE} ( θ , φ , x ) = {𝔼}_{{q}_{φ} ( z \vert x )} [ log {p}_{θ } ( x \vert z ) ] - ( 1 - α ) {D}_{KL} ( {q}_{φ} ( z \vert x ) \vert \vert {p}_{θ} ( z ) ) - ( α + λ - 1 ) {D}_{MMD} ( {q}_{φ} ( z ) \vert \vert {p}_{θ} ( z ) ) )$$

where α and λ are hyperparameters, weighting the mutual information and agreement with the chosen latent feature space distribution respectively. The final term may be the maximum-mean discrepancy divergence, which is computed and valid.

### Design of the Encoder and Decoder

To implement a variational autoencoder, a parameterized encoder, q_{φ}(z|x) 102, and decoder, pθ(x|z) 104, is provided herein. The encoder 102 and decoder 104 design may include enhancements, over a generic design, to improve its function on protein sequence data. In the particular case of encoding protein sequences, the data distribution may be expected to be highly complex, in the sense of having many different interactions between amino acids. Whichever model that is used to estimate the joint distribution over amino acids should be sufficient to express every proteomic device that is known to exist. Additionally, the model should be able to capture interactions between amino acids distant in sequence space. This may be due to the one-dimensional protein sequence representing a protein embedded in three dimensions. In order to have a useful model, both accurate reconstruction and a useful latent space are desired. These specifications are addressed by the design considerations herein. Due to the complexity of the distribution attempting to be estimated, an assumption that the model will benefit from a very deep ResNet style convolutional network may be adopted. In one embodiment, distant interactions between residues are addressed by using dilated convolutions. Advantageously, application of dilated convolutions may allow for exponential increase in the receptive field of the network.

In one embodiment, the chosen network architecture has a receptive field large enough to capture dependencies between any pair of amino acids in the input sequence. To free the autoencoder model from memorizing the fine details of the model (e.g., the particular amino acid distribution of a beta sheet) the decoder 104 may be augmented with an autoregressive module 106. The autoregressive module 106 can learn the local structure of the amino acid sequence, leaving the latent space to encode the higher level details, such as secondary structure into the feature space. Combining the design considerations leads to the architecture visualized in FIG. 1.

Specifically, the encoder 102 may contain some number (e.g., 25 in one embodiment) of convolutional ResNet blocks with some number (e.g., two in another embodiment) of strided convolution layers for downscaling and channel doubling. The dilation pattern may repeat every five blocks, for example. Any other number of blocks and any other pattern of repetition may be used. In one embodiment, the decoder 104 may reverse the encoder structure. Inside of each module, the cubes each represents a layer type. Layers 101 indicate a one-dimensional convolutional layer with skip connections in the style of ResNet. In one embodiment, layers 101 may have progressively larger dilation within a single repetition. Patterned layers 103 indicate a one-dimensional convolution where the length of the input is halved with a stride of two and the channels are doubled. Patterned layers 105 indicate the reverse operation of patterned layers 103 via a transposed one-dimensional convolution.

### Training

To train this model, the cleaned SwissProt database may be used, for example. In other embodiments, any other suitable database may be used. In one embodiment, all of these elements may be combined and the model may be trained end-to-end using the ADAM optimizer, for example. In other embodiments, any other suitable optimizer may be used.

### Protein Property Inference

Once an instance of BioSeqVAE is trained, the latent feature space may be used to predict the phenotype of a given protein. This task may be performed using a supervised learning approach. A dataset relating sequence to function may be provided in order to learn which points in latent feature space relate to specific functions. It is worth noting that this can be done for any imaginable protein property for which a dataset can be gathered. Some possible properties include Gene Ontology IDs, temperature stability, EC Number, or protein localization. In practice, much of the required data is gathered and is readily available across many bioinformatics databases.

In one embodiment, supervised models may be created by using BioSeqVAE to encode all protein sequences in the data set into a latent feature vector. Then that latent feature vector and the associated phenotype is used to train the model. In one embodiment, a random forest model from scikit-learn may be used without parameter tuning for training. When both the unsupervised variational autoencoding model and a set of supervised phenotype models are created, targeted design of function becomes possible.

### Protein Design

Using BioSeqVAE, the design problem may be reduced to a search of the latent feature space, as every point in the space may be associated with a protein sequence that is likely to fold and have some function. In one embodiment, the design task relies on down-stream models to predict how points in the latent feature space relate to desirable phenotypes. In one embodiment, a set of models that relate points in the latent feature space to different phenotypes {ƒᵢ}^{N}i=1, can be leveraged to generate enzymes with any combination desired properties. This allows design to be rephrased as an optimization problem in Euclidean space as follows:

$${max}_{z} {\sum }_{i = 1}^{m} {{α}_{i} ( {f}_{i} ( z ) - {c}_{i} )}^{2} \qquad ( 2 )$$

where ƒᵢ is the ith model, cᵢ is the target (e.g., a specific sequence length), and αᵢ is a weight. Once solved, the optimal point in latent feature space, \hat{ }z, is decoded to find a candidate protein to test in downstream experiments.

### Results

One of BioSeqVAE's capabilities is to encode protein sequences into an information rich latent feature space and generate protein sequences that are likely to fold and function. Analyses may first be performed to validate the models core function. BioSeqVAE, once trained, has a multitude of downstream uses. Good downstream performance on an enzyme classification task and a protein homology regression task is demonstrated, then how the model can be used to design new sequences is provided. The intent of these tasks is to demonstrate that the latent feature space encodes features that are useful for downstream learning rather than chasing state of the art performance on each task. The ultimate objective is to develop models that allow the user to find points in the latent feature space that generate proteins with properties of interest. To emphasize this point, representative sequences for each of the models presented are generated. The section is culminated by producing sequences that are likely to have a combination of desirable properties.

### Model Validation

To validate the model is performing correctly, both qualitative and quantitative methods are employed. As an overall performance measure, the accuracy of encoding and then decoding the same protein is evaluated. Then, the distribution of the latent feature space is estimated to check that it is close a standard normal distribution. Random samples are sampled from the latent feature space and decoded to show qualitatively that the generated sequences look correct. Finally, a well characterized protein is reconstructed and tested to see that its reconstruction is likely to retain function.

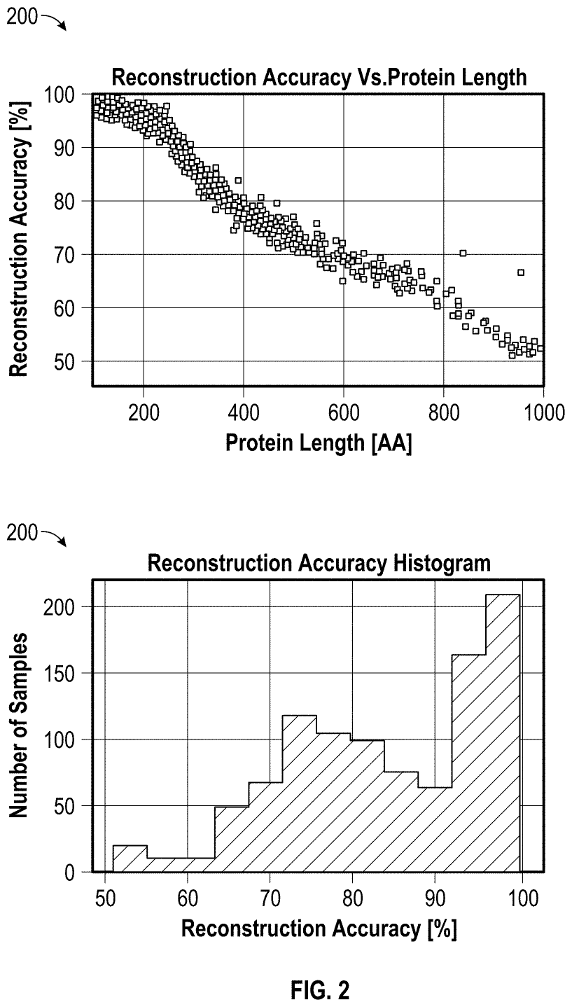

FIG. 2 is a block diagram 200 that illustrates that BioSeqVAE is able to reconstruct proteins in the test set with accuracy that is dependent on length. It is very effective at reconstructing short proteins and the accuracy trails off to around 50% at 1000 amino acids. On average, reconstruction accuracy is 83.7%±12.1%.

In one embodiment, BioSeqVAE decodes proteins accurately from the latent feature space. To test this, known proteins from the test set are embedded using the encoder, then decoded to reconstruct the original sequence. 1000 proteins from the test set are reconstructed. The percent agreement between the actual sequence and the predicted reconstruction is calculated. The results of this test are visualized in FIG. 2. The average reconstruction accuracy is 83.7%±12.1%. The length of the protein is related to the reconstruction accuracy, with the algorithm performing better on shorter proteins. For proteins with less than 200 amino acids, greater than 90% reconstruction accuracy may be expected. One method to improve reconstruction accuracy across the board may be to increase the dimension of the latent feature space.

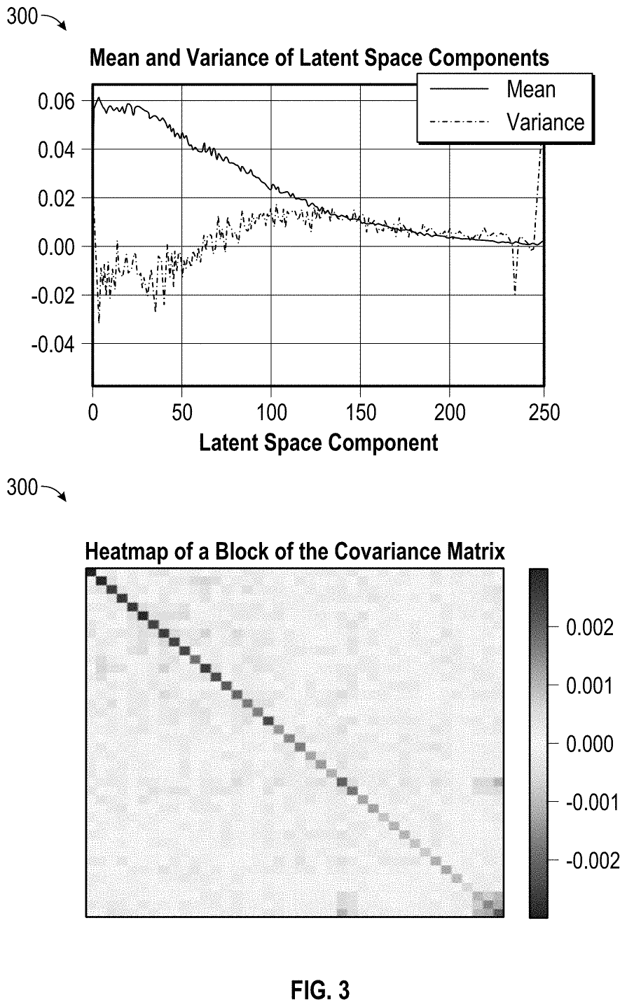

The latent feature space can be sampled from easily, and produces qualitatively valid random samples. In one embodiment, to validate that the feature space can produce good protein sequence samples, 10,000 proteins from the test set are encoded into the feature space. The mean and covariance matrix for those encoded features is calculated. Then, latent feature space samples are drawn from a multivariate normal with the estimated statistics. The KL divergence term in the loss encourages the latent feature space to have a standard normal distribution. In practice, that exact distribution may be approximated. The mean and covariance matrix are visualized in FIG. 3. One thing to notice is that the diagonal components of the covariance matrix are largest, showing that the model disentangles features from the data set into a set of features that are closer to independent.

As shown in the block diagram 300 FIG. 3, the latent space can be well approximated as a multivariate Gaussian with 250 dimensions. The dimensions of the Gaussian are close to independent. Using the mean and covariance matrix efficient samples representative of protein sequences can be synthesized. Samples of random protein sequences are obtained by sampling from the latent feature space, then running them through the BioSeqVAE decoder. Qualitatively, one can see that these proteins do not have any obvious artifacts such as long amino acid repeats. When these sequences are BLASTed against UniProt database, they have small stretches of homology with sequences that were not in the training set demonstrating that they share qualities with known proteins.

>Random Protein 1 example:
MAAIPEELYEAVNDASSRFVSVHEEQKSQLDLMMFSDRMVRVKSEAAHHTS
MTNIEIYLKWEQMGQQSVMSVRQTSPLGLVNQFQAFATPIDAAFDRLENAL
RLTSLLMQGGPIDNRDRDGLLINVNYDAHGAEADGNLEAAASSASSFACPQ
AMLDTYSGPITKLLLQVNHLPVSPIILKADGLANLFWHIFVSMRFFTSIVH
PLLLFIYYPLILGPLFEAQVPIRWPTFSVLEASYAMYHLEDPVSSLLEFSK
AMALICYSCLGNSFILHDHPLHYERVAFNSGFVWGNLHLLASSL
>Random Protein 2 example:
MGRLDAADVILADFGTQIVDVGAPRTKGQVEMVSVLLLHLDDPHGPIRASL
GENSLDFTSPTDQLLLSPDESSVTALLLPTYLLGPVHQPAHRGGNLLLLTA
APNTRKSFPDASHTPMSHTMLDEKLKMMTREETTRDFGQRENLHEYIKNYA
TQYKRQTIGAVKHKNEQFESKEDWSIQQMNDGGISMFTSSAYANKSIPPGS
SEAPLTESIAFLKNTAVSRAIMNPRQVNPFETIKKLEYSKKVRLNEEEPDV
FNKAKLNGVKMSLNESKDSLGRPQKYPINPNAREYVNSREGLPHSLIPKHR
LSFQDVGSLETHNDTMPVSLGNSIEQYAAVDAQRDDLRISEFSKDPKLFSA
DIDCEKEICNAMAASDLLDIWGFYAEAESKQNEGLGYILKQLPIRHLCRHS
DRIIEIRGKRAPSYTVGLFASLFQCLVEFTFAPLVSTQDASSALPITQQRD
EQLISVYCKVFQQQTVLEKFKQEIVWDNLKMFKDSWVTCLCVFLIPEEKKV
VTTRMGYSALSNLQSRDQCFFSTLADMKIWVFPADSSRHHMKPT
>Random Protein 3 example:
MTPAKKPKMSEVWDYAVGQITALSQVPEDGLPVCLGWDGGWRTSGNERVTI
VELQPEAANGLAGSSTLPLQDWSWNRERDVAATQLLLRAATGAEATMSPNN
LNRGKASALCLQYLTPNFTSFLAYAVSQDHALLQA

### Generation of Representative Examples of Proteins with a Desired Phenotype

One capability of BioSeqVAE's is to encode protein sequences into an information rich latent feature space and generate protein sequences that are likely to fold and function. If sampling randomly from the latent feature space, one cannot be sure of the phenotype of the protein sequence that is generated. In order to learn the relationship between points in the latent feature space and phenotype of interest, supervised learning may be performed on smaller subsets of data. This relationship may be easiest for the model to learn if BioSeqVAE encodes informative features. A phenotype model can be used to predict which points in the latent feature space correspond to proteins of interest. From those points in the latent space, BioSeqVAE can hallucinate syntactically valid proteins that are likely to have the desired phenotype. In this way the strengths of two separate models may be paired and used for design and/or phenotypic inference.

### Enzyme Type can be Accurately Determined from Protein Sequence

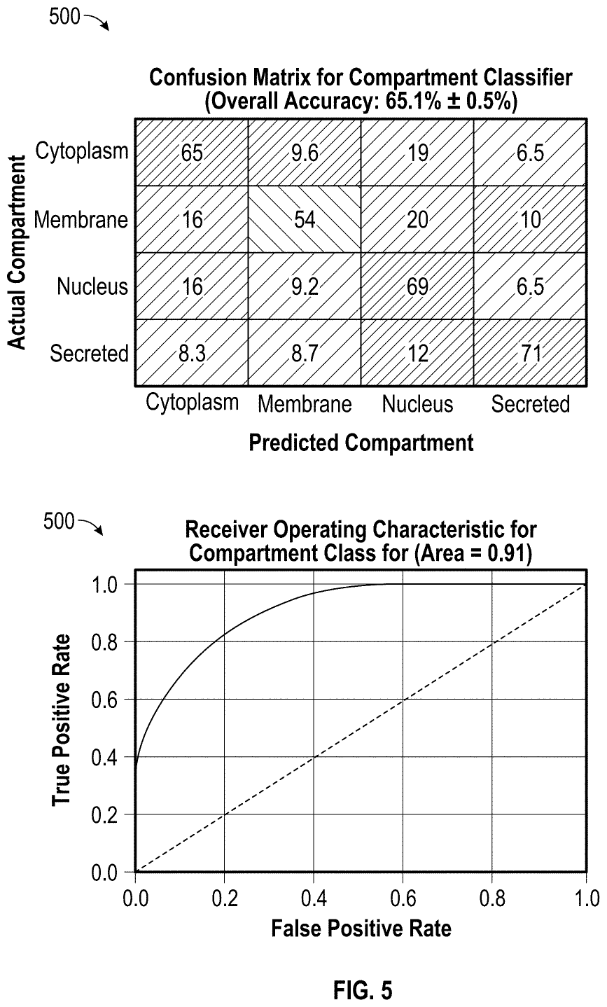

In one embodiment, a simple random forest classification model from scikit-learn may be applied to a dataset of 60000 proteins obtained from the UniProt database where both sequence and EC Number were known. In one embodiment, the protein sequences were encoded into a 250 dimensional vector of features using BioSeqVAE and these features and the top level EC Number were used in a supervised learning setting to train a random forest classifier. In this case, the classifier achieved 70.6% cross validated error (see FIG. 5).

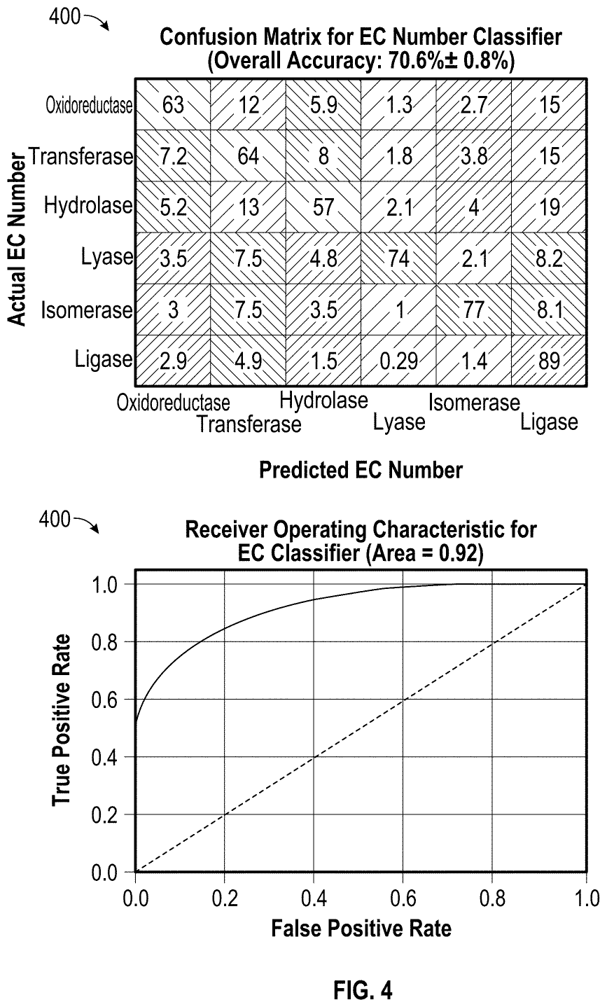

Referring to the block diagram 400 of FIG. 4, the latent feature space can be used to both determine which enzyme class that a sequence belongs to and create novel examples of that type of enzyme. In one embodiment, a balanced dataset of 60000 examples of enzymes with known EC numbers were used to train this model. The confusion matrix shows how well each class is predicted from a given point in the latent space. The combined receiver operating characteristic curve for the classifier. Using the model above and the technique outlined herein, representative sequences from each class of proteins were generated. When blasted against the UniProt database they show homology with proteins of their designated type.

>Hallucinated Oxidoreductase, Confidence 35%
example:
MIDPGEVTPKRAGAQKEQFGLIHRPMKPVDVALTSANQPKEFDASVKDSRG
GGQRTLIRGDKPRCDWKVVRVEQEALSDILYTGTDASLQAVLDEDRRFYEL
AEFRKNRVRDILEDEPVSGQFFEQQDKINTGNKHTMAVAATGFDSFCMIAG
AEEMIASGMPIGSARYKQQRYQGGFIEANGNESQLNGLHHLTSPVAMRCTP
PMDMMAFPDDDGKQFMKGNPILPFDLGLGRKWASLTAFAGRAAARTAEGFH
QGVD
>Hallucinated Transferase, Confidence 31% example:
MSSSAGRKSTKVDYPFLLSTSCDTEYYLGMAAVFRDLDKHGRAAHDVVVKA
RGELAQRGILDERKSARDSFPIILLITLGPVMKEASLYPIQLIDFPLALNP
EAKHAWVLHPLEHREPYGPVYPTLEAAGLPALGSVTVKLRCPAATTVEKIY
IIQTGFEVAQQLNANVSTSPGMIWHARNSAPAMVVDQENILQGAPGKSTAL
IQTYYDSGGWIGDRFSEPKKVFHGRAAPNDNPKLLASFPLQLLMLVAVAND
KSWNIEMAARGADYTAAGDAACSDVVGAATGGAIKGLPSEKRLLLNAGSTG
ERLATMADVLTTPGTAAMGIAADAPLYGAATGAVNDQRFFHEKVGAYPATT
RAADETLTPQLQYEAGDLLSKKALAYDISAASYEACSVVFLLASRLHLAAA
ISGHLGAQFMELDPLSYNEAISALNFQAFHQREISAWLWRRQFLIGP
>Hallucinated Hydrolase, Confidence 37% example:
MTASPKNRQNVYNPQFNDIEEISPVEVKSSHKIIGSHNAEVNFKNVRTDEA
KQSYFIEIFENVSFYYEDGSEDAAFFEYPIKHLLKKPTSAARECGGDWLAK
EEVLEFPLSTRYIEAGRDLDLQDGPLVPSVPGFARGQSPIEPNDFDEFLSF
GLGITKSMHTEKSNEVGNAAFNFFKSIYDRYYGSYRRDHGSGVPAYIVRRW
IPLGSGARIISRTSAIGTVISFVYSSMTYVDSEITFMGADRQAGFRARVNP
LRFDIYCDARPIHKPDPSQSLHFAPDYLAEQAKITVVRRPHDQGIVYEGGL
KAIVAAITFCKPFDFLSSNIYDWILKRATPVIALNDGGISGAFLLLDPHPK
DDQHDRVHLKLGFAATIQLYAAEIEWAYRIQNLHEHAYFEIL
>Hallucinated Lyase, Confidence 26% example:
MVRSEVKGFDGGRSPRRKLRRGKRGAVILIEGLVCQAVAGAVPGIARGPKG
QLLAPATATASAAIAIFVLSGFYVPPGHWLTVSHHAAQAFFAVTDADNFLQ
RVRVRYRTQLYMLDIPRRMRMNPMAGATYLGETAADSAFENATQTGEMCAF
AVVPIISLGRRSWPLSNVWIGTTVAAEVPALGLAARAWNVQIRSAAAGDLY
PCYLYDTKDPPFDLYLMILAQILLDIPGQAAAVLAAIKRERLLLVQRLGTA
ALKA
>Hallucinated Isomerase, Confidence 27% example:
MQATLRRQYKGPKEVVEGALLMRLAEAGFCWAGVWGRKTVVVDGRADAGVH
LARILGLPEVRASEGVWAMLMLRPRLRDYLIKRIDRSPTYVQQPRLRASGA
EREGQALAKSEDSAAKAPDYYKGPFDLDNHISETLEASYSKEATGHPSGHP
GAPWTAPADSPGGANDRDKPAHEIMTHREDLATTPAQTFQRLEEGALVYLL
LEAAALQRGQL
>Hallucinated Ligase, Confidence 56% example:
MEKERLMYPVMHDSIQMGDAASGQRDTHMIHQGPFAFRRIRVQQEKPYYRS
DDESYAISKLERPSPQISRQGDVACSTEARPPDSVFLSGAADSGTVCAKVA
ASGKGARNNEMKGLFGQVKELSPNAKVGLVFLKVRLAREPDSFRWTRQGGD
DVALDLPRELIDRIGQTVDLLRKQPVNIPIGKERCRIDAIYQAGQYNVWQL
GLVCMGCGQYFYRVKGTEAKRIYVDISLSASVTISVCEGYAHRDGMANDDT
GVTSVVAIFRLPTRILDYAAARMTRQLSWPAPVDRATVDTDDDLEAILLYL
LLVLNPYTYFPGPFWAVCVLRLWAGASTGMQILLGQAATDLLQYYEGMGTV
YLKNNANVIIFRKLLCGMHKRYLYDI

Referring to the block diagram 500 of FIG. 5, the latent feature space can be used to both determine which enzyme class that a sequence belongs to and create novel examples of that type of enzyme. A balanced dataset of 60000 examples of enzymes with known EC numbers were used to train this model. The confusion matrix shows how well each class is predicted from a given point in the latent space. The combined receiver operating characteristic curve for the classifier. Using the supervised compartment prediction model and the technique outlined herein Representative sequences localized to the following compartments were generated.

>Hallucinated Cytoplasm Protein, Confidence 47%
example:
MQKQLYTGIIIEIVNLVLPNLHVTYILKACSETEIVPCAVHLDMVAGEGVS
ELPRTIATLSCSMTFEKYGMGRMSAGYDIPICVDAYPNSFSFLRWWDNLLD
KLEGVLEIMSNLYDGFEISPYKISPAIIPRETQTEDETYDKATARGVFHVN
VCYQMIQFESTGDRALMIDRLAVTAMLQSLGIMAHAFASWNFDPGMVGQVG
LDGAPVGGHTFKAKHEKSSGSFDTLQAAGEIFSQWIPPIPDIHGSLSTIWW
AFAAVIAAGSGFCYYLLMCARVAASIIQDRLLLFRDAYVIAGLAATTNVYP
WDYFMNDTVQKAAPYAAHGLLALPVIMLIYWLLLELIYAML
>Hallucinated Membrane Protein, Confidence 54%
example:
MYASRRGSLYLRLVSQLQARDSHQRGAYSIVKYPPYTTAKLATAASIMDSK
LAKVHDLRLLDVYFNNPYNEQKFHAVMQAIEIELTGCIRQGFESQGQDQNR
YILNGPSGVELKGTFSGLLYIDYLYLYHVTKGHNPLDFTERRAGIHVINFF
HQLDTYSAATRARAAVLHNSAANFQINHNNKIGSWLCKDCQIPSTPHHATF
LGDLKERGPRMPRQALQAGARKVVELNDHNSGFICEGAHSEKATWVTASHP
LDYLRKLLWHESLSSFLDAANQLLQTVGDSHKHPLLAFLLLSVSAWVLHNQ
LPSFRVRYNRFILLFSQLRAAPNIPCECFVLKQISIKKFRLIRPRYARYAI
HGGILAALPDHARKNKWVNNQEKLENGHFVAAQHDVPREAGEL
>Hallucinated Nucleus Protein, Confidence 76%
example:
MAASSHPRPQCERSWLNRGQPAETASREFFLRYGKPFLCEAPRAGVFGHCL
QDQTSGQMESGGMSSVTEAAELFASGIAKWVSIVIRQPSVSSHFVNPLLVA
SWADRGLSVGKSIVTLEARYDKEVLEPVVECNRSNALEGAISPSEEYNDND
LSLNESINGKGIKELGHPTSGRAEEYLLYFPDTASKSVIVKSLSKMDVETI
YCFIENPARLTSQSFTCMWTALSIQARVAAEYIGFLFLQTHYDSWDLTL
>Hallucinated Secreted Protein, Confidence 63%
example:
MLAFLLRPLLILVFAAGTSMARAGPRLPPPIGSKGSSECSSFISDCDNRVY
TFEDEIRHARESAPVNSKPSEYLHRVQGHEAEQDEQFFNPASEVSACEIGA
VGLMAERANVHGASVLCPAKAQYLALPIYLPFTGHTYVGAFQDERWASFCP
MNTAGQVNVIYKTSDGDSQIELLIIRMAKHQSAAVVASYGSEKKLKRAQGH
HTAESTNNQLISIQMIQSTGEVVGSLTTSTAAIPKYISTGLTVGRKESLTA
AFAGAALEAYISATRLALAANNWYHPPFDWGKHRDDMVQL

### Homology Between Pairs of Protein Sequences can be Rapidly and Accurately Predicted from the Latent Space

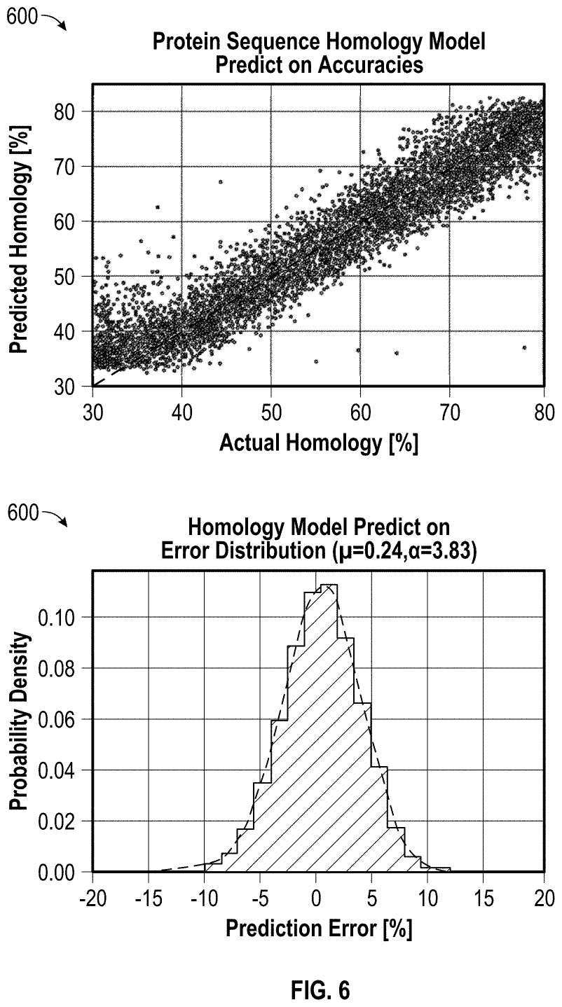

To test the usefulness of the latent space for regression tasks, in one embodiment a random forest regression model implemented in scikit-learn may be used to learn homology from latent space embedding. In one embodiment, 14,000 pairs of protein sequences were taken from the SwissProt database. The homology percent of each pair was calculated. From that database a model was created relating both latent space embeddings to the homology percent. The resulting model had an error standard deviation of 3.83%. FIG. 6 illustrates the cross-validated performance of that model in block diagram 600.

### The Latent Space Can Be Leveraged to Design Proteins

In one embodiment, several models from above may be combined to synthesize protein sequences that are likely to possess multiple functions of interest. First, the conversion of a membrane protein into a protein localized in the cytosol is demonstrated. Second, enzymes creation with a set homology from a starting enzyme of interest is demonstrated. In one embodiment, a model may be created to detect compartment and then a high homology protein may be created that switches compartment.

As demonstrated herein, realistic protein sequences can be hallucinated from an unsupervised machine learning model, BioSeqVAE. The properties of sequences can be intuited from the latent feature space of BioSeqVAE. This opens up the possibility to use much larger and easier-to-collect datasets and leverage those for the creation of novel proteins for an array of applications. Disclosed herein is a novel way to tackle pathway completion when looking for proteins in pathways for orphaned metabolites. Hyperparameter optimization may be performed on BioSeqVAE to maximize the performance of this model before experimental work.

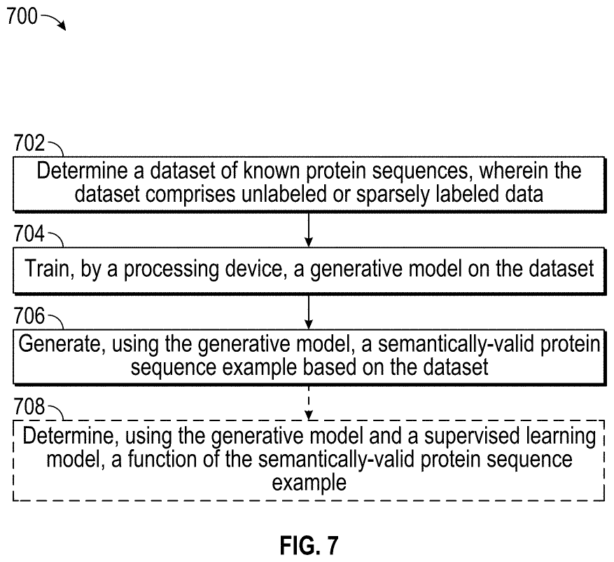

FIG. 7 is a flow diagram 700 illustrating unsupervised protein sequence generation, according to one embodiment. The method 700 may be performed by processing logic that comprises hardware (e.g., circuitry, dedicated logic, programmable logic, microcode, etc.), software (e.g., instructions run on a processing device to perform hardware simulation), or a combination thereof.

Referring to FIG. 7, processing logic at block 702 determines a dataset of known protein sequences. In one embodiment, the dataset includes unlabeled and/or sparsely labeled data. In one embodiment, the dataset is a subset of known protein sequences from a complete dataset of known protein sequences, wherein the subset is determined based on selecting a defined number of protein sequences from each cluster of the complete dataset. In another embodiment, the dataset is a complete dataset.

At block 704, processing logic trains, by a processing device, a generative model on the dataset and, at block 706, processing logic generates, using the generative model, a semantically-valid protein sequence example based on the dataset. In various embodiments, the generative model is capable of analyzing protein sequences of variable lengths, modelling interactions between distant amino acid residues, utilizing a latent feature space, and generating realistic protein sequences, among other capabilities.

Optionally, at block 708, processing logic determines, using the generative model and a supervised learning model, a function of the semantically-valid protein sequence example. In one embodiment, determining the function includes predicting a phenotype of the semantically-valid protein sequence by inputting a point, associated with the semantically-valid protein sequence, in a latent feature space of the generative model into the supervised learning model.

In one embodiment, the supervised learning model is trained to determine protein sequence function by encoding, using the generative model, the dataset of known protein sequences into a latent feature vector, and training the supervised learning model on the latent feature vector and an associated phenotype. In one embodiment, the processing logic may use the generative model and the supervised model to generate a protein sequence having a target phenotype, as described herein.

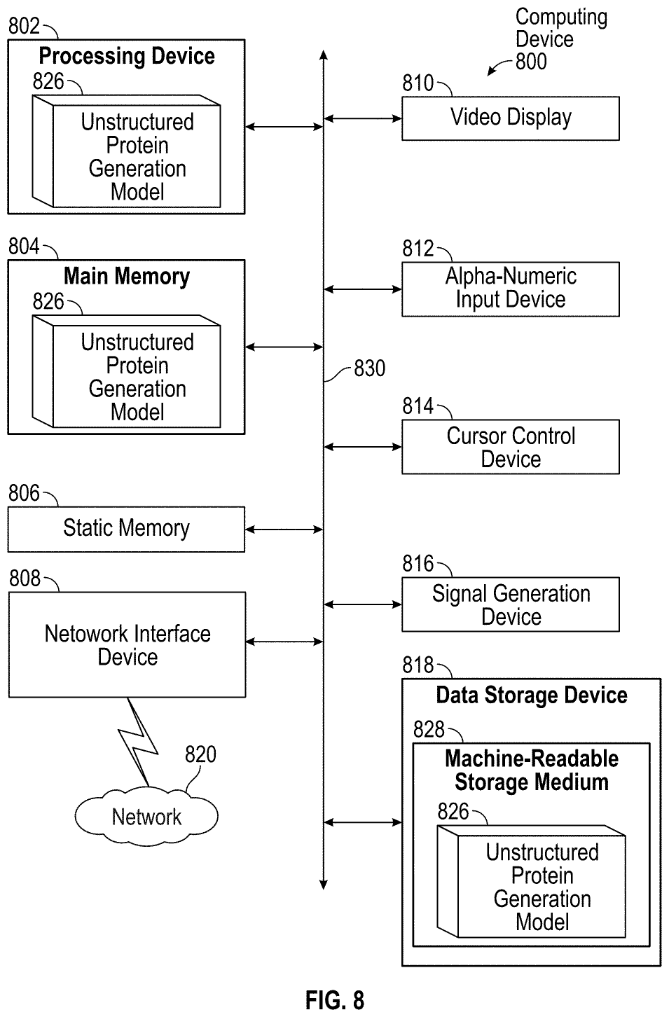

FIG. 8 is a block diagram of an example apparatus that may perform one or more of the operations described herein, in the example form of a computer system 800 within which a set of instructions, for causing the machine to perform any one or more of the methodologies discussed herein, may be executed. In alternative embodiments, the machine may be connected (e.g., networked) to other machines in a local area network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a server, a network router, a switch or bridge, a hub, an access point, a network access control device, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The exemplary computer system 800 includes a processing device 802, a main memory 804 (e.g., read-only memory (ROM), flash memory, dynamic random access memory (DRAM), a static memory 806 (e.g., flash memory, static random access memory (SRAM), etc.), and a data storage device 818, which communicate with each other via a bus 830. Any of the signals provided over various buses described herein may be time multiplexed with other signals and provided over one or more common buses. Additionally, the interconnection between circuit components or blocks may be shown as buses or as single signal lines. Each of the buses may alternatively be one or more single signal lines and each of the single signal lines may alternatively be buses.

Processing device 802 represents one or more general-purpose processing devices such as a microprocessor, central processing unit, or the like. More particularly, the processing device may be complex instruction set computing (CISC) microprocessor, reduced instruction set computer (RISC) microprocessor, very long instruction word (VLIW) microprocessor, or processor implementing other instruction sets, or processors implementing a combination of instruction sets. Processing device 802 may also be one or more special-purpose processing devices such as an application specific integrated circuit (ASIC), a field programmable gate array (FPGA), a digital signal processor (DSP), network processor, or the like. The processing device 802 is configured to execute processing logic/instructions (e.g., unstructured protein generation model) 826, for performing the operations and steps discussed herein.

The data storage device 818 may include a non-transitory machine-readable storage medium 828, on which is stored one or more set of logic/instructions (e.g., unstructured protein generation model) 826 (e.g., software) embodying any one or more of the methodologies of functions described herein, including instructions to cause the processing device 802 to execute operations described herein. The logic/instructions (e.g., unstructured protein generation model) 826 may also reside, completely or at least partially, within the main memory 804 or within the processing device 802 during execution thereof by the computer system 800; the main memory 804 and the processing device 802 also constituting machine-readable storage media. The logic/instructions (e.g., unstructured protein generation model) 826 may further be transmitted or received over a network 820 via the network interface device 808.

While the non-transitory machine-readable storage medium 828 is shown in an exemplary embodiment to be a single medium, the term “machine-readable storage medium” should be taken to include a single medium or multiple media (e.g., a centralized or distributed database, or associated caches and servers) that store the one or more sets of instructions. A machine-readable medium includes any mechanism for storing information in a form (e.g., software, processing application) readable by a machine (e.g., a computer). The machine-readable medium may include, but is not limited to, magnetic storage medium (e.g., floppy diskette); optical storage medium (e.g., CD-ROM); magneto-optical storage medium; read-only memory (ROM); random-access memory (RAM); erasable programmable memory (e.g., EPROM and EEPROM); flash memory; or another type of medium suitable for storing electronic instructions.

The preceding description sets forth numerous specific details such as examples of specific systems, components, methods, and so forth, in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular embodiments may vary from these exemplary details and still be contemplated to be within the scope of the present disclosure.

Additionally, some embodiments may be practiced in distributed computing environments where the machine-readable medium is stored on and or executed by more than one computer system. In addition, the information transferred between computer systems may either be pulled or pushed across the communication medium connecting the computer systems.

Embodiments of the claimed subject matter include, but are not limited to, various operations described herein. These operations may be performed by hardware components, software, firmware, or a combination thereof.

Although the operations of the methods herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent or alternating manner.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

It will be appreciated that variants of the above-disclosed and other features and functions, or alternatives thereof, may be combined into may other different systems or applications. Various presently unforeseen or unanticipated alternatives, modifications, variations, or improvements therein may be subsequently made by those skilled in the art which are also intended to be encompassed by the following claims. The claims may encompass embodiments in hardware, software, or a combination thereof

### References

Kevin K Yang, Zachary Wu, and Frances H Arnold. Machine learning in protein engineering. arXiv preprintarXiv:1811.10775, 2018.
Toshihiro Nakashima, Hitoshi Toyota, Itaru Urabe, and Tetsuya Yomo. Effective selection system for experimental evolution of random polypeptides towards DNA-binding protein. J Biosci Bioeng, 103(2):155-160, February 2007.
Pengfei Tian and Robert B Best. How many protein sequences fold to a given structure? a coevolutionary analysis. Biophys J, 113(8):1719-1730, October 2017.
Justin B Siegel, Amanda Lee Smith, Sean Poust, Adam J Wargacki, Arren Bar-Even, Catherine Louw, Betty WShen, Christopher B Eiben, Huu M Tran, Elad Noor, Jasmine L Gallaher, Jacob Bale, Yasuo Yoshikuni, Michael HGelb, Jay D Keasling, Barry L Stoddard, Mary E Lidstrom, and David Baker. Computational protein design enables a novel one-carbon assimilation pathway. Proc Natl Acad Sci USA, 112(12):3704-3709, March 2015.
Hans Renata, Z Jane Wang, and Frances H Arnold. Expanding the enzyme universe: accessing non-natural reactions by mechanism-guided directed evolution. Angew Chem Int Ed Engl, 54(11):3351-3367, March 2015.
Devin L Trudeau, Toni M Lee, and Frances H Arnold. Engineered thermostable fungal cellulases exhibit efficient synergistic cellulose hydrolysis at elevated temperatures. Biotechnol Bioeng, 111(12):2390-2397, December 2014.
Daniel-Adriano Silva, Shawn Yu, Umut Y. Ulge, Jamie B. Spangler, Kevin M. Jude, Carlos Labão-Almeida,Lestat R. Ali, Alfredo Quijano-Rubio, Mikel Ruterbusch, Isabel Leung, Tamara Biary, Stephanie J. Crowley, Enrique Marcos, Carl D. Walkey, Brian D. Weitzner, Fátima Pardo-Avila, Javier Castellanos, Lauren Carter, Lance Stewart, Stanley R. Riddell, Marion Pepper, Gonçalo J. L. Bernardes, Michael Dougan, K. Christopher Garcia, and David Baker. De novo design of potent and selective mimics of IL-2 and IL-15.Nature, 565(7738):186-191, January 2019.
Viktor Stein and Kirill Alexandrov. Synthetic protein switches: design principles and applications. TrendsBiotechnol, 33(2):101-110, February 2015.
Justin B Siegel, Alexandre Zanghellini, Helena M Lovick, Gert Kiss, Abigail R Lambert, Jennifer L St Clair, Jasmine L Gallaher, Donald Hilvert, Michael H Gelb, Barry L Stoddard, Kendall N Houk, Forrest E Michael, and David Baker. Computational design of an enzyme catalyst for a stereoselective bimolecular diels-alder reaction. Science, 329(5989):309-313, July 2010.
Frances H Arnold. Directed evolution: bringing new chemistry to life. Angew Chem Int Ed Engl, 57(16):4143-4148, April 2018.[11]Andrew R Buller, Paul van Roye, Jackson K B Cahn, Remkes A Scheele, Michael Herger, and Frances H Arnold. Directed evolution mimics allosteric activation by stepwise tuning of the conformational ensemble. J Am ChemSoc, 140(23):7256-7266, June 2018.[12]Po-Ssu Huang, Scott E Boyken, and David Baker. The coming of age of de novo protein design.Nature,537(7620):320-327, September 2016.[13] Ivan Coluzza. Computational protein design: a review. J Phys Condens Matter, 29(14):143001, April 2017.[14]The UniProt Consortium. UniProt: the universal protein knowledgebase. Nucleic Acids Res, 46(5):2699, March 2018.
Helen M Berman, John Westbrook, Zukang Feng, Gary Gilliland, Talapady N Bhat, Helge Weissig, Ilya NShindyalov, and Philip E Bourne. The protein data bank. Nucleic acids research, 28(1):235-242, 2000.[16]Tero Karras, Timo Aila, Samuli Laine, and Jaakko Lehtinen. Progressive growing of GANs for improved quality, stability, and variation. arXiv, October 2017.
Diederik P. Kingma and Prafulla Dhariwal. Glow: Generative flow with invertible 1×1 convolutions. arXiv, July 2018.9
Chin-Wei Huang, David Krueger, Alexandre Lacoste, and Aaron Courville. Neural autoregressive flows. arXiv, April 2018.
Jiajun Wu, Chengkai Zhang, Tianfan Xue, Bill Freeman, and Josh Tenenbaum. Learning a probabilistic latent space of object shapes via 3d generative-adversarial modeling. In D. D. Lee, M. Sugiyama, U. V. Luxburg, I. Guyon, and R. Garnett, editors, Advances in Neural Information Processing Systems 29, pages 82-90. Curran Associates, Inc., 2016.
Romain Lopez, Jeffrey Regier, Michael B Cole, Michael I Jordan, and Nir Yosef.

Deep generative modeling for single-cell transcriptomics. Nat Methods, 15(12):1053-1058, December 2018.
Mikel Artetxe, Gorka Labaka, and Eneko Agirre. Unsupervised statistical machine translation. arXiv, September 2018.
Jyh-Jing Hwang, Sergei Azernikov, Alexei A. Efros, and Stella X. Yu. Learning beyond human expertise with generative models for dental restorations. arXiv, March 2018.[23] Diederik P Kingma and Max Welling. Auto-encoding variational bayes. arXiv, December 2013.
Ian Goodfellow, Jean Pouget-Abadie, Mehdi Mirza, Bing Xu, David Warde-Farley, Sherjil Ozair, Aaron Courville, and Yoshua Bengio. Generative adversarial nets. In Z. Ghahramani, M. Welling, C. Cortes, N. D. Lawrence, and K. Q. Weinberger, editors, Advances in Neural Information Processing Systems 27, pages 2672-2680. Curran Associates, Inc., 2014.
Laurent Dinh, David Krueger, and Yoshua Bengio. NICE: Non-linear independent components estimation. arXiv, October 2014.
Laurent Dinh, Jascha Sohl-Dickstein, and Samy Bengio. Density estimation using real NVP. arXiv, May 2016.
Akosua Busia, George E. Dahl, Clara Fannjiang, David H. Alexander, Elizabeth Dorfman, Ryan Poplin, Cory Y. McLean, Pi-Chuan Chang, and Mark DePristo. A deep learning approach to pattern recognition for short DNAsequences. BioRxiv, June 2018.
Jakob Nybo Nissen, Casper Kaae Sonderby, Jose Juan Almagro Armenteros, Christopher Heje Groenbech, Henrik Bjorn Nielsen, Thomas Nordahl Petersen, Ole Winther, and Simon Rasmussen. Binning microbial genomes using deep learning. BioRxiv, December 2018.
Adam J Riesselman, John B Ingraham, and Debora S Marks. Deep generative models of genetic variation capture the effects of mutations. Nat Methods, 15(10):816-822, October 2018.
Martin Steinegger and Johannes Soding. Clustering huge protein sequence sets in linear time. Nat Commun,9(1):2542, June 2018.
Shengjia Zhao, Jiaming Song, and Stefano Ermon. Info VAE: Information maximizing variational autoencoders. arXiv, June 2017.
Shengjia Zhao, Jiaming Song, and Stefano Ermon. The information autoencoding family: A lagrangian perspective on latent variable generative models. arXiv preprint arXiv:1806.06514, 2018.
Ishaan Gulrajani, Kundan Kumar, Faruk Ahmed, Adrien Ali Taiga, Francesco Visin, David Vazquez, and Aaron Courville. Pixel VAE: A latent variable model for natural images arXiv, November 2016.
Kaiming He, Xiangyu Zhang, Shaoqing Ren, and Jian Sun. Identity mappings in deep residual networks. InBastian Leibe, Jiri Matas, Nicu Sebe, and Max Welling, editors, Computer vision—ECCV 2016, volume 9908 of Lecture notes in computer science, pages 630-645. Springer International Publishing, Cham, 2016.
Kaiming He, Xiangyu Zhang, Shaoqing Ren, and Jian Sun. Deep residual learning for image recognition. In The IEEE Conference on Computer Vision and Pattern Recognition (CVPR), June 2016.[36]Fisher Yu, Vladlen Koltun, and Thomas Funkhouser. Dilated residual networks. In 2017 IEEE Conference on Computer Vision and Pattern Recognition (CVPR), pages 636-644. IEEE, July 2017.[37] Fisher Yu and Vladlen Koltun. Multi-scale context aggregation by dilated convolutions. arXiv, November 2015.
Tim Salimans, Andrej Karpathy, Xi Chen, and Diederik P. Kingma. PixelCNN++: Improving the PixelCNN with discretized logistic mixture likelihood and other modifications. arXiv, January 2017.
Aaron van den Oord, Nal Kalchbrenner, Oriol Vinyals, Lasse Espeholt, Alex Graves, and Koray Kavukcuoglu. Conditional image generation with Pixel CNN decoders. arXiv, June 2016.
Aaron van den Oord, Nal Kalchbrenner, and Koray Kavukcuoglu. Pixel recurrent neural networks. arXiv, January 2016.
Aaron van den Oord, Sander Dieleman, Heiga Zen, Karen Simonyan, Oriol Vinyals, Alex Graves, Nal Kalch-brenner, Andrew Senior, and Koray Kavukcuoglu. WaveNet: A generative model for raw audio. arXiv, September 2016.
Nal Kalchbrenner, Lasse Espeholt, Karen Simonyan, Aaron van den Oord, Alex Graves, and Koray Kavukcuoglu. Neural machine translation in linear time. CoRR, abs/1610.10099, 2016.
Diederik P. Kingma and Jimmy Ba. Adam: A method for stochastic optimization.CoRR, abs/1412.6980, 2014.
F. Pedregosa, G. Varoquaux, A. Gramfort, V. Michel, B. Thirion, O. Grisel, M. Blondel, P. Prettenhofer, R. Weiss,V. Dubourg, J. Vanderplas, A. Passos, D. Cournapeau, M. Brucher, M. Perrot, and E. Duchesnay. Scikit-learn: Machine learning in Python.Journal of Machine Learning Research, 12:2825-2830, 2011.

What is claimed is:

1. A method of unsupervised protein sequence generation, comprising:
determining a dataset of known protein sequences, wherein the dataset comprises unlabeled or sparsely labeled data;
training, by a processing device, a generative model on the dataset; and
generating, using the generative model, a semantically-valid protein sequence example based on the dataset.

2. The method of claim 1, wherein the dataset is a subset of known protein sequences from a complete dataset of known protein sequences, wherein the subset is determined based on selecting a defined number of protein sequences from each cluster of the complete dataset.

3. The method of claim 1, further comprising determining, using the generative model and a supervised learning model, a function of the semantically-valid protein sequence example.

4. The method of claim 3, wherein determining the function comprises predicting a phenotype of the semantically-valid protein sequence by inputting a point, associated with the semantically-valid protein sequence, in a latent feature space of the generative model into the supervised learning model.

5. The method of claim 3, wherein the supervised learning model is trained by:
encoding, using the generative model, the dataset of known protein sequences into a latent feature vector; and
training the supervised learning model on the latent feature vector and an associated phenotype.

6. The method of claim 1, wherein the generative model is to analyze protein sequences of variable lengths, model interactions between distant amino acid residues, utilize a latent feature space, and generate realistic protein sequences.

7. The method of claim 1, further comprising generating, using the generative model and a supervised model, a protein sequence having a target phenotype.

8. A variational autoencoder for unsupervised protein sequence generation, comprising:
a parameterized encoder to estimate a latent variable in a latent space given a particular data point in data space; and
a decoder to produce an output in the data space given a particular point in the latent space, wherein the decoder is augmented with an autoregressive module to learn a local structure of an amino acid sequence.

9. The variational autoencoder of claim 8, the parameterized encoder comprising a plurality of convolutional ResNet blocks.

10. The variational autoencoder of claim 9, the parameterized encoder further comprising a one-dimensional convolution layer, in which a length of an input to the parameterized encoder is halved which a stride of two, and a channel associated with the parameterized encoder is doubled.

11. The variational autoencoder of claim 9, wherein each of the plurality of convolutional ResNet blocks comprises a plurality of strided convolution layers for downscaling and channel doubling.

12. The variational autoencoder of claim 11, wherein a dilation pattern of the plurality of strided convolution layers repeats every five blocks.

13. The variational autoencoder of claim 8, the decoder comprising a plurality of convolutional ResNet blocks.

14. The variational autoencoder of claim 13, the decoder further comprising a first one-dimensional convolution layer, transposed with respect to a second one-dimensional convolution layer of the parameterized encoder.

15. The variational autoencoder of claim 13, each of the plurality of convolutional ResNet blocks comprising a plurality of strided convolution layers.

16. The variational autoencoder of claim 15, wherein a dilation pattern of the plurality of strided convolution layers repeats every five blocks.

17. The variational autoencoder of claim 15, wherein a first pattern of the plurality of strided convolution layers of the decoder is opposite a second pattern of a plurality of strided convolution layers of the parameterized encoder.

18. The variational autoencoder of claim 15, wherein the parameterized encoder and the decoder are deep learning models parameterized by respective weights.

19. The variational autoencoder of claim 8, wherein the variational autoencoder is to:
determine a dataset of known protein sequences, wherein the dataset comprises unlabeled or sparsely labeled data;
train a generative model on the dataset; and
generate, using the generative model, a semantically-valid protein sequence example based on the dataset.

20. The variational autoencoder of claim 19, wherein the variational autoencoder is further to determine, using the generative model and a supervised learning model, a function of the semantically-valid protein sequence example.


SEQUENCE LISTING (13 sequences)

SEQ ID NO: 1 - protein, 299 aa, Artificial Sequence
  Random Protein 1
MAAIPEELYEAVNDASSRFVSVHEEQKSQLDLMMFSDRMVRVKSEAAHHTSMTNIEIYLKWEQMGQQSVMSVRQTSPLGLVNQFQAFATPIDAAFDRLENALRLTSLLMQGGPIDNRDRDGLLINVNYDAHGAEADGNLEAAASSASSFACPQMLDTYSGPAITKLLLQVNHLPVSPIILKADGLANLFWHIFVSMRFFTSIVHPLLLFIYYPLILGPLFEAQVPIRWPTFSVLEASYAMYHLEDPVSSLLEFSKAMALICYSCLGNSFILHDHPLHYERVAFNSGFVWGNLHLLASSL

SEQ ID NO: 2 - protein, 554 aa, Artificial Sequence
  Random Protein 2
MGRLDAADVILADFGTQIVDVGAPRTKGQVEMVSVLLLHLDDPHGPIRASLGENSLDFTSPTDQLLLSPDESSVTALLLPTYLLGPVHQPAHRGGNLLLLTAAPNTRKSFPDASHTPMSHTMLDEKLKMMTREETTRDFGQRENLHEYIKNYATQYKRQTIGAVKHKNEQFESKEDWSIQQMNDGGISMFTSSAYANKSIPPGSSEAPLTESIAFLKNTAVSRAIMNPRQVNPFETIKKLEYSKKVRLNEEEPDVFNKAKLNGVKMSLNESKDSLGRPQKYPINPNAREYVNSREGLPHSLIPKHRLSFQDVGSLETHNDTMPVSLGNSIEQYAAVDAQRDDLRISEFSKDPKLFSADIDCEKEICNAMAASDLLDIWGFYAEAESKQNEGLGYILKQLPIRHLCRHSDRIIEIRGKRAPSYTVGLFASLFQCLVEFTFAPLVSTQDASSALPITQQRDEQLISVYCKVFQQQTVLEKFKQEIVWDNLKMFKDSWVTCLCVFLIPEEKKVVTTRMGYSALSNLQSRDQCFFSTLADMKIWVFPADSSRHHMKPT

SEQ ID NO: 3 - protein, 137 aa, Artificial Sequence
  Random Protein 3
MTPAKKPKMSEVWDYAVGQITALSQVPEDGLPVCLGWDGGWRTSGNERVTIVELQPEAANGLAGSSTLPLQDWSWNRERDVAATQLLLRAATGAEATMSPNNLNRGKASALCLQYLTPNFTSFLAYAVSQDHALLQA

SEQ ID NO: 4 - protein, 259 aa, Artificial Sequence
  Hallucinated Oxidoreductase, Confidence 35%
MIDPGEVTPKRAGAQKEQFGLIHRPMKPVDVALTSANQPKEFDASVKDSRGGGQRTLIRGDKPRCDWKVVRVEQEALSDILYTGTDASLQAVLDEDRRFYELAEFRKNRVRDILEDEPVSGQFFEQQDKINTGNKHTMAVAATGFDSFCMIAGAEEMIASGMPIGSARYKQQRYQGGFIEANGNESQLNGLHHLTSPVAMRCTPPMDMMAFPDDDGKQFMKGNPILPFDLGLGRKWASLTAFAGRAAARTAEGFHQGVD

SEQ ID NO: 5 - protein, 455 aa, Artificial Sequence
  Hallucinated Transferase, Confidence 31%
MSSSAGRKSTKVDYPFLLSTSCDTEYYLGMAAVFRDLDKHGRAAHDVVVKARGELAQRGILDERKSARDSFPIILLITLGPVMKEASLYPIQLIDFPLALNPEAKHAWVLHPLEHREPYGPVYPTLEAAGLPALGSVTVKLRCPAATTVEKIYIIQTGFEVAQQLNANVSTSPGMIWHARNSAPAMVVDQENILQGAPGKSTALIQTYYDSGGWIGDRFSEPKKVFHGRAAPNDNPKLLASFPLQLLMLVAVANDKSWNIEMAARGADYTAAGDAACSDVVGAATGGAIKGLPSEKRLLLNAGSTGERLATMADVLTTPGTAAMGIAADAPLYGAATGAVNDQRFFHEKVGAYPATTRAADETLTPQLQYEAGDLLSKKALAYDISAASYEACSVVFLLASRLHLAAAISGHLGAQFMELDPLSYNEAISALNFQAFHQREISAWLWRRQFLIGP

SEQ ID NO: 6 - protein, 399 aa, Artificial Sequence
  Hallucinated Hydrolase, Confidence 37%
MTASPKNRQNVYNPQFNDIEEISPVEVKSSHKIIGSHNAEVNFKNVRTDEAKQSYFIEIFENVSFYYEDGSEDAAFFEYPIKHLLKKPTSAARECGGDWLAKEEVLEFPLSTRYIEAGRDLDLQDGPLVPSVPGFARGQSPIEPNDFDEFLSFGLGITKSMHTEKSNEVGNAAFNFFKSIYDRYYGSYRRDHGSGVPAYIVRRWIPLGSGARIISRTSAIGTVISFVYSSMTYVDSEITFMGADRQAGFRARVNPLRFDIYCDARPIHKPDPSQSLHFAPDYLAEQAKITVVRRPHDQGIVYEGGLKAIVAAITFCKPFDFLSSNIYDWILKRATPVIALNDGGISGAFLLLDPHPKDDQHDRVHLKLGFAATIQLYAAEIEWAYRIQNLHEHAYFEIL

SEQ ID NO: 7 - protein, 259 aa, Artificial Sequence
  Hallucinated Lyase, Confidence 26%
MVRSEVKGFDGGRSPRRKLRRGKRGAVILIEGLVCQAVAGAVPGIARGPKGQLLAPATATASAAIAIFVLSGFYVPPGHWLTVSHHAAQAFFAVTDADNFLQRVRVRYRTQLYMLDIPRRMRMNPMAGATYLGETAADSAFENATQTGEMCAFAVVPIISLGRRSWPLSNVWIGTTVAAEVPALGLAARAWNVQIRSAAAGDLYPCYLYDTKDPPFDLYLMILAQILLDIPGQAAAVLAAIKRERLLLVQRLGTAALKA

SEQ ID NO: 8 - protein, 215 aa, Artificial Sequence
  Hallucinated Isomerase, Confidence 27%
MQATLRRQYKGPKEVVEGALLMRLAEAGFCWAGVWGRKTVVVDGRADAGVHLARILGLPEVRASEGVWAMLMLRPRLRDYLIKRIDRSPTYVQQPRLRASGAEREGQALAKSEDSAAKAPDYYKGPFDLDNHISETLEASYSKEATGHPSGHPGAPWTAPADSPGGANDRDKPAHEIMTHREDLATTPAQTFQRLEEGALVYLLLEAAALQRGQL

SEQ ID NO: 9 - protein, 383 aa, Artificial Sequence
  Hallucinated Ligase, Confidence 56%
MEKERLMYPVMHDSIQMGDAASGQRDTHMIHQGPFAFRRIRVQQEKPYYRSDDESYAISKLERPSPQISRQGDVACSTEARPPDSVFLSGAADSGTVCAKVAASGKGARNNEMKGLFGQVKELSPNAKVGLVFLKVRLAREPDSFRWTRQGGDDVALDLPRELIDRIGQTVDLLRKQPVNIPIGKERCRIDAIYQAGQYNVWQLGLVCMGCGQYFYRVKGTEAKRIYVDISLSASVTISVCEGYAHRDGMANDDTGVTSVVAIFRLPTRILDYAAARMTRQLSWPAPVDRATVDTDDDLEAILLYLLLVLNPYTYFPGPFWAVCVLRLWAGASTGMQILLGQAATDLLQYYEGMGTVYLKNNANVIIFRKLLCGMHKRYLYDI

SEQ ID NO: 10 - protein, 347 aa, Artificial Sequence
  Hallucinated Cytoplasm Protein, Confidence 47%
MQKQLYTGIIIEIVNLVLPNLHVTYILKACSETEIVPCAVHLDMVAGEGVSELPRTIATLSCSMTFEKYGMGRMSAGYDIPICVDAYPNSFSFLRWWDNLLDKLEGVLEIMSNLYDGFEISPYKISPAIIPRETQTEDETYDKATARGVFHVNVCYQMIQFESTGDRALMIDRLAVTAMLQSLGIMAHAFASWNFDPGMVGQVGLDGAPVGGHTFKAKHEKSSGSFDTLQAAGEIFSQWIPPIPDIHGSLSTIWWAFAAVIAAGSGFCYYLLMCARVAASIIQDRLLLFRDAYVIAGLAATTNVYPWDYFMNDTVQKAAPYAAHGLLALPVIMLIYWLLLELIYAML

SEQ ID NO: 11 - protein, 399 aa, Artificial Sequence
  Hallucinated Membrane Protein, Confidence 54%
MYASRRGSLYLRLVSQLQARDSHQRGAYSIVKYPPYTTAKLATAASIMDSKLAKVHDLRLLDVYFNNPYNEQKFHAVMQAIEIELTGCIRQGFESQGQDQNRYILNGPSGVELKGTFSGLLYIDYLYLYHVTKGHNPLDFTERRAGIHVINFFHQLDTYSAATRARAAVLHNSAANFQINHNNKIGSWLCKDCQIPSTPHHATFLGDLKERGPRMPRQALQAGARKVVELNDHNSGFICEGAHSEKATWVTASHPLDYLRKLLWHESLSSFLDAANQLLQTVGDSHKHPLLAFLLLSVSAWVLHNQLPSFRVRYNRFILLFSQLRAAPMPCECFVLKQISIKKFRLIRPRYARYAIHGGILAALPDHARKNKWVNNQEKLENGHFVAAQHDVPREAGEL

SEQ ID NO: 12 - protein, 251 aa, Artificial Sequence
  Hallucinated Nucleus Protein, Confidence 76%
MAASSHPRPQCERSWLNRGQPAETASREFFLRYGKPFLCEAPRAGVFGHCLQDQTSGQMESGGMSSVTEAAELFASGIAKWVSMRQPSVSSHFVNPLLVASWADRGLSVGKSIVTLEARYDKEVLEPVVECNRSNALEGAISPSEEYNDNDLSLNESINGKGIKELGHPTSGRAEEYLLYFPDTASKSVIVKSLSKMDVETIYCFIENPARLTSQSFTCMWTALSIQARVAAEYIGFLFLQTHYDSWDLTL

SEQ ID NO: 13 - protein, 295 aa, Artificial Sequence
  Hallucinated Secreted Protein, Confidence 63%
MLAFLLRPLLILVFAAGTSMARAGPRLPPPIGSKGSSECSSFISDCDNRVYTFEDEIRHARESAPVNSKPSEYLHRVQGHEAEQDEQFFNPASEVSACEIGAVGLMAERANVHGASVLCPAKAQYLALPIYLPFTGHTYVGAFQDERWASFCPMNTAGQVNVIYKTSDGDSQIELLIIRMAKHQSAAVVASYGSEKKLKRAQGHHTAESTNNQLISIQMIQSTGEVVGSLTTSTAAIPKYISTGLTVGRKESLTAAFAGAALEAYISATRLALAANNWYHPPFDWGKHRDDMVQL